(19) United States
(12) Patent Application Publication
(10) Pub. No.: US 2024/0358553 A1
(43) Pub. Date: Oct. 31, 2024

(54) DYNAMICALLY ADJUSTABLE COMPRESSION GARMENT FOR A POST-OPERATIVE, POST-PARTUM, AND/OR OBESE PATIENT

(71) Applicant: Nandhika Wijay, Houston, TX (US)

(72) Inventor: Nandhika Wijay, Houston, TX (US)

(21) Appl. No.: 18/308,823

(22) Filed: Apr. 28, 2023

(51) Int. Cl. A41D 13/12

(52) U.S. Cl. CPC A61F 13/148; A61F 5/03

(57) ABSTRACT

The invention provides a dynamically adjustable compression garment for a post-operative, post-partum, and/or obese patient that is properly structured, dimensioned, and configured to apply an appropriate compressive force to surgically treated portions of a patient's body, and that can be adjusted in real time as the patient is wearing it, particularly to change a direction/vector of compression. More specifically, embodiments of the present invention relate to a compression garment with tension straps configured to attach to the garment as opposed to other tension straps, whereby a direction/vector of compression can be adjusted dynamically.

### FIELD OF THE INVENTION

The present invention relates generally to a dynamically adjustable compression garment removably disposed on a post-operative, post-partum, and/or obese patient. More specifically, embodiments of the present invention relate to a compression garment with tension straps configured to attach to the garment as opposed to other tension straps, whereby a direction/vector of compression can be adjusted dynamically.

### BACKGROUND OF THE INVENTION

The purpose of a compression garment is to support the surgical site, the incisions, and the affected skin. If the compression garment is too tight, it affects circulation and hinders healing. A compression garment should only exert gentle, supportive pressure, so it should lay flat and smooth on the skin.

Post-surgical compression garments assist healing processes and improve post-operative results of reconstructive or aesthetic (plastic) surgery procedures such as augmentation, lipostructure (fat-transfer liposuction and lipofilling), that disturb the cohesion between the dermis, subcutaneous fat, and muscle layers. In particular, such procedures bruise connective tissues, traumatize capillaries and blood vessels, and compromise the lymphatic fluid distribution system. Serum and blood seep into adjacent tissues, causing swelling, lymphedema, and inflammation, all of which, if not constrained will stretch the epidermis and dermis skin layers affecting adherence of the affected dermal layers to the underlying tissues.

U.S. Pat. No. 8,784,347B1 provides a post-surgery, high-back, compression garment for post-operatively immobilizing surgically corrected tissues to ensure proper healing and provide molded gluteal-shaping power-net domes that preserve desired anatomic curvature enhancements and corrections in the gluteal regions of the human body during convalescence following contouring gluteoplasty procedures.

U.S. Pat. No. 8,105,256B1 provides a compression garment for treating post-operative patients involved in body contouring surgery and is structured for removable disposition in an operative position about the torso or other predetermined portion of the patient's body.

The compression garment is dimensioned, configured, and structured to apply a predetermined, appropriate compression to the surgically affected portions of the patient in order to reduce pain, diminish swelling and avoid fluid accumulation, while not impairing the venous and lymphatic circulation of the affected body portion. The garment includes a base of flexible, compressible material having at least one, but more practically both a first and second cover member overlying the internal and external surfaces of the base, wherein a closure assembly serves to removably maintain the garment in a variably closed orientation while in the operative position such that appropriate compressive force is applied to the affected areas including adjacent soft tissue and bony prominences.

U.S. Pat. No. 6,936,021B1 provides a close-fitting, compression garment for providing adjustable compressive force over a surgical site in the dorsocervical region of the human body below the neck, above, and between the shoulder blades of the upper back. The vest body portion of the invented garment has graduated or increasing elasticity encircling the upper torso and shoulder region of the body. Wide adjustable elastic pressure bands pressure anchored at the dorsocervical apex at the back of the vest stretch over the shoulders of the patient and are adjustably anchored to anchor strips secured to the front chest surface of the vest for establishing compression over a surgical site in the dorsocervical region during recovery as needed both for rehabilitation and comfort.

Available post-surgical compression garments fail to consider variations in patients' bodies, including but not limited to bony areas or prominences such as the iliac crest and/or the rib cage area. These areas of bony prominences, when subjected to the same compressive force as soft tissue areas, are frequently subjected to abrasions and other injuries due to excessively high pressure being applied to these bony portions. In contrast, soft tissue portions of a patient's body, including the central part of the abdomen, are generally subjected to lower and sometimes inadequate compressive forces. This is due to a “tent” effect at least partially caused by the adjacent or surrounding bony prominences engaging the force-generating structure applied to the patient's body. Accordingly, certain portions of the patient's body are subjected to lower amounts of compression and are more prone to develop complications such as fluid retention and fibrosis. Furthermore, traditional compression garments are not optimized for post-partum and/or obese patients.

Therefore, there is a need for a compression garment for a post-operative, post-partum, and/or obese patient that is properly structured, dimensioned, and configured to apply an appropriate compressive force to surgically treated portions of a patient's body, and that can be adjusted in real time as the patient is wearing it. Such a dynamically adjustable compression garment for a post-operative, post-partum, and/or obese patient should be applied during a post-operative period so as to alleviate and/or significantly reduce discomfort to the patient by the reduction of pain, the diminishing of swelling, and/or the avoidance of fluid accumulation, as generally set forth above.

### SUMMARY OF THE INVENTION

To address the foregoing problems, in whole or in part, and/or other problems that may have been observed by persons skilled in the art, the present disclosure provides compositions and methods as described by way of example as set forth below.

An improved dynamically adjustable compression garment for a post-operative, post-partum, and/or obese patient is provided that can change the direction/vector of compression on a patient's body as needed. In one embodiment, the compression garment comprises a front portion, a rear portion, a torso extension having a lower edge, a lower edge, and a pair of arm openings, the front portion, the rear portion, and the torso extension each having an interior surface and an exterior surface. The compression garment further comprises a front crotch opening cutout, elastic thigh seams respectively joining the front and back thigh section edges of the left and of the right thigh sections forming left and right thigh compression sleeves, and at least four diagonally arranged tension straps attached to a spine portion of the compression garment configured to provide compression at a target body area of the patient, and wherein the tension straps are configured to attach to the compression garment as opposed to other tension straps, whereby a direction/vector of compression can be adjusted dynamically. The at least four diagonally arranged tension straps comprise a first tension strap having a first female portion of a hook and loop closure (e.g., VELCRO®) on a distal end of the first tension strap, a second tension strap having a second female portion of the hook and loop closure on a distal end of the second tension strap, a third tension strap having a first male portion of the hook and loop closure on a proximal end of the third tension strap, a fourth tension strap having a second male portion of the hook and loop closure on a proximal end of the fourth tension strap, a third female hook and loop closure portion attached on the right thigh compression sleeve and a fourth female hook and loop closure portion attached on the left thigh compression sleeve and the at least four diagonally arranged tension straps are configured to securely lock the female portions of the hook and loop closure with the male portions of the hook and loop closure.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 illustrates a schematic front view of the dynamically adjustable compression garment for a post-operative, post-partum, and/or obese patient, in accordance with an embodiment of the present invention; and

FIG. 2 illustrates a schematic back view of the dynamically adjustable compression garment for a post-operative, post-partum, and/or obese patient, in accordance with an embodiment of the present invention.

Skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF THE INVENTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

The present invention provides a dynamically adjustable compression garment for a post-operative, post-partum, and/or obese patient that is configured to enable changing a direction/vector of compression by having uniquely designed tension straps capable of attaching to left and right thigh compression sleeves of the compression garment. A “male” hook and loop closure portion (e.g., VELCRO®) is located on the tension strap and a “female” hook and loop closure portion is on the left and right thigh compression sleeves. This unique design allows for the ability to change the direction of a compression force on a target area of the patient.

In an embodiment of the present invention, the dynamically adjustable compression garment for a post-operative, post-partum, and/or obese patient is configured to wrap around a patient's torso and comprises two adjustable shoulder straps configured to be rested around the shoulders of the patient.

In another embodiment of the present invention, the dynamically adjustable compression garment for a post-operative, post-partum, and/or obese patient is made of a moisture-wicking material, and comprises a closure mechanism which is water-resistant and prevents water from entering to the inside of the garment. Also, there is provided a zipper on the front side of the compression garment for donning and doffing the compression garment by the patient.

The tension straps are sewn to the spine of the garment. The tension straps are angled in a way to avoid injury by avoiding placing pressure on the buttocks of the patient after surgery. In one embodiment, the portion of the dynamically adjustable compression garment that covers the patient's buttocks comprises a softer material than the rest of the compression garment. The pubic region is the most difficult place to apply pressure after surgery, and the present invention is able to apply such pressure safely and effectively by having uniquely designed tension straps that attach to the garment specifically on the thigh.

The compression garment of the present invention may be manufactured and provided in various sizes to accommodate different individuals. A suitable size of the compression garment is able to provide an appropriate compressive force to the torso or other appropriate portion of the user's body depending on the size and shape of the engaged body portion.

In an embodiment of the present invention, the structural and operative features of the garment assembly facilitate a compressive force being applied in a manner that will accommodate the bony prominences of the body, through the application of an appropriately lesser compressive force, while concurrently or simultaneously applying an appropriately greater compressive force to the soft tissue portions. As a result, the compression garment of the present invention will serve to reduce pain and recovery time by diminishing swelling in a post-operative patient while preventing the occurrence of bruises or other skin abrasions.

In an exemplary embodiment, the garment may be used at the hospital and/or home, enabling a patient to shower and sponge bath with secure placement of post-surgical drains and freedom of movement for both arms, while maintaining light compression for the surgical area, and protection from direct water contact to prevent bacteria and infection in the surgical area or the area surrounding drains.

In another exemplary embodiment, the garment is worn around the torso, shoulders, and/or waist of the patient. In another exemplary embodiment, the garment is worn around portions of the patient's body that include the patient's arms, legs, thighs, calves, and knees.

In another exemplary embodiment, the garment is made of a poly-Lycra fabric blend, spandex, cotton, nylon, or other breathable/moisture-wicking material that is worn for daily use to protect a surgical wound and secure post-surgical drains and tubing. The compression garment is formed of an at least partially elastic material so as to define the compression zone on the patient's body.

In an embodiment of the present invention, the compression garment comprises one or more straps and is closed using a secure closure sewn into the compression garment, such as a hook and loop closure (e.g., VELCRO®), zipper, or other secure closure. In another exemplary embodiment, the closure is water resistant and prevents water from entering the garment through the front closure.

In an exemplary embodiment, the side of the compression garment may have an opening, for example, on the right and left side of the garment. In an exemplary embodiment, each opening is large enough to allow the post-surgical drain to be inserted therethrough, for example approximately two inches in length. Each opening can be covered, for example, via sewn on flaps. In an exemplary embodiment, the flaps are made of water-resistant material.

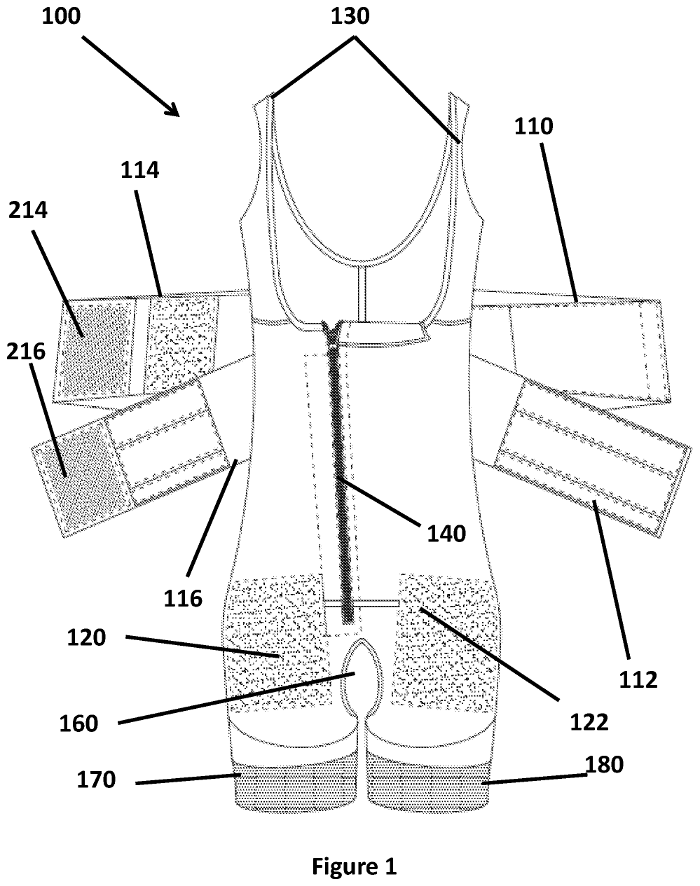

FIG. 1 illustrates a schematic front view of the dynamically adjustable compression garment for a post-operative, post-partum, and/or obese patient in accordance with an embodiment of the present invention. The dynamically adjustable compression garment for a post-operative, post-partum, and/or obese patient 100 for a patient as shown in FIG. 1 shows a front crotch opening cutout 160, elastic thigh seams respectively joining the front and back thigh section edges of the left and of the right thigh sections forming left and right thigh compression sleeves 180, 170, at least four diagonally arranged tension straps attached to a spine portion of the compression garment 100 configured to provide compression at a target body area of the patient. FIG. 1 shows a third tension strap 114 having a first male portion 214 of the hook and loop closure on a proximal end of the third tension strap 114, a fourth tension strap 116 having a second male portion 216 of the hook and loop closure on a proximal end of the fourth tension strap 116, a third female hook and loop closure portion 120 attached on the right thigh compression sleeve 170 and a fourth female hook and loop closure portion 122 attached on the right thigh compression sleeve 180. The figure further shows at least two adjustable shoulder straps 130 configured to be rested around the shoulders of the patient, a zipper 140 for donning and doffing of the compression garment 100. The tension straps are configured to attach to the compression garment as opposed to other tension straps, whereby a direction/vector of compression can be adjusted dynamically.

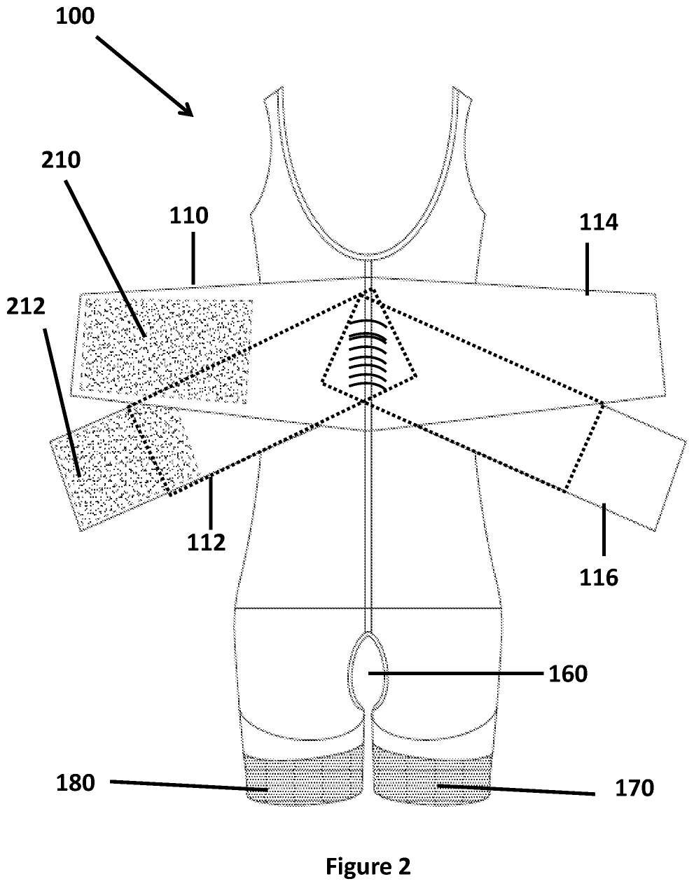

FIG. 2 illustrates a schematic back view of the dynamically adjustable compression garment for a post-operative, post-partum, and/or obese patient in accordance with an embodiment of the present invention. The dynamically adjustable compression garment for a post-operative, post-partum, and/or obese patient 100 for a patient as shown in FIG. 2 shows a front crotch opening cutout 160, at least four diagonally arranged tension straps attached to a spine portion of the compression garment 100, and a first tension strap 110 having a first female portion 210 of a hook and loop closure on a distal end of the first tension strap 110, and a second tension strap 112 having a second female portion 212 of the hook and loop closure on a distal end of the second tension strap 112.

In an embodiment of the present invention, the at least four diagonally arranged tension straps are configured to securely lock the female portions 210, 212, 120, 122 of the hook and loop closure with the male portions 214, 216 of the hook and loop closure.

In an embodiment of the present invention, the dynamically adjustable compression garment for a post-operative, post-partum, and/or obese patient 100 comprises one or more flaps that are attached to the front portion of the compression garment at a top of a corresponding one or more openings in the compression garment and are further secured by a flap closure to the front portion on a bottom and on each side of the corresponding one of the one or more openings. The one or more flaps prevent water from entering the inside of the compression garment through each of the one or more openings.

In an embodiment of the present invention, the dynamically adjustable compression garment for a post-operative, post-partum, and/or obese patient 100 comprises one or more of a post-surgical drain, a catheter, a colostomy bag, a collection bag, a collection container, and tubing. In further embodiments, the dynamically adjustable compression garment for a post-operative, post-partum, and/or obese patient 100 comprises at least one and preferably one or more pairs of slits formed therein to allow passage of the post-surgical drainage tubes to the exterior of the compression garment 100. The location of the slits is guided by the location of the surgical site and the site of the incision. The slits can be formed at locations that are at least partially around a side of the patient (i.e., one end of the slit is located on the front of the garment, while the other end can be located along the side of the patient). When there are two pairs of slits, the slits are spaced apart but can be formed parallel to one another. The slits can also be formed at an angle relative to the bottom edge of the garment. In other words, the slits are not parallel to the bottom edge. The spacing between slits on one side is sufficient to allow passage of the drainage tubes therefrom to the bulbs.

Some of the non-limiting advantages of the present invention include:
1. Provides necessary compression for promoting and ensuring proper skin adhesion.
2. Adjusts compression as swelling increases or decreases to maintain constant comfort.
3. Controls swelling.
4. Helps skin better fit its new contours.
5. Easy to put on, adjust, and remove during the healing process.
6. Maintains surgical dressings in place until removed.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof, and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. Although the foregoing subject matter has been described in some detail by way of illustration and example for purposes of clarity of understanding, it will be understood by those skilled in the art that certain changes and modifications can be practiced within the scope of the appended claims.

What is claimed:

1. A dynamically adjustable compression garment for a post-operative, post-partum, and/or obese patient for a patient, comprising:
a. a front portion, a rear portion, a torso extension having a lower edge, a lower edge and a pair of arm openings, the front portion, the rear portion, and the torso extension each having an interior surface and an exterior surface;
b. a front crotch opening cut-out;
c. elastic thigh seams respectively joining the front and back thigh section edges of the left and of the right thigh sections forming left and right thigh compression sleeves;
d. at least four diagonally arranged tension straps attached to a spine portion of the compression garment configured to provide compression at a target body area of the patient, and wherein the tension straps are configured to attach to the compression garment as opposed to other tension straps, whereby a direction/vector of compression can be adjusted dynamically, and wherein the at least four diagonally arranged tension straps comprise:
i. a first tension strap having a first female portion of a hook and loop closure on a distal end of the first tension strap;
ii. a second tension strap having a second female portion of the hook and loop closure on a distal end of the second tension strap;
iii. a third tension strap having a first male portion of the hook and loop closure on a proximal end of the third tension strap; and
iv. a fourth tension strap having a second male portion of the hook and loop closure on a proximal end of the fourth tension strap; and
e. a third female hook and loop closure portion attached on the right thigh compression sleeve; and
f. a fourth female hook and loop closure portion attached on the right thigh compression sleeve;
wherein the at least four diagonally arranged tension straps are configured to securely lock the female portions of the hook and loop closures with the male portions of the hook and loop closures.

2. The dynamically adjustable compression garment for a post-operative, post-partum, and/or obese patient of claim 1, configured to wrap around the patient's torso.

3. The dynamically adjustable compression garment for a post-operative, post-partum, and/or obese patient of claim 1, comprising at least two adjustable shoulder straps configured to be rested around the shoulders of the patient.

4. The dynamically adjustable compression garment for a post-operative, post-partum, and/or obese patient of claim 1, comprising a moisture-wicking material.

5. The dynamically adjustable compression garment for a post-operative, post-partum, and/or obese patient of claim 1, comprising a closure mechanism that is water resistant.

6. The dynamically adjustable compression garment for a post-operative, post-partum, and/or obese patient of claim 1, comprising a zipper for putting on and taking off the compression garment by the patient.

7. The dynamically adjustable compression garment for a post-operative, post-partum, and/or obese patient of claim 1, comprising one or more flaps attached to the front portion of the compression garment at a top of one or more corresponding openings in the compression garment, and secured to the front portion by a flap closure on a bottom and on each side of the one or more corresponding openings.

8. The dynamically adjustable compression garment for a post-operative, post-partum, and/or obese patient of claim 7, wherein each of the one or more flaps is configured to prevent water from entering the inside of the compression garment through one or more of the one or more corresponding openings.

9. The dynamically adjustable compression garment for a post-operative, post-partum, and/or obese patient of claim 1, comprising one or more of a post-surgical drain, a catheter, a colostomy bag, a collection bag, a collection container, and a tubing.

10. The dynamically adjustable compression garment for a post-operative, post-partum, and/or obese patient of claim 1, comprising an at least partially elastic material configured to define a compression zone on the patient's body.
